(19) United States
(12) Patent Application Publication
(10) Pub. No.: US 2006/0216694 A1
(43) Pub. Date: Sep. 28, 2006

(54) Use of a virus expressing a binding moiety to measure analytes in a sample

(76) Inventors: David James Squirrell, Salisbury, Wiltshire (GB); Martin Alan Lee, Wiltshire (GB); Carl Nicholas Mayers, Wiltshire (GB)

Correspondence Address: JOHN S. PRATT, ESQ;KILPATRICK STOCKTON, LLP, 1100 PEACHTREE STREET, ATLANTA, GA, 30309, US

(21) Appl. No.: 10/548,111

(22) PCT Filed: Mar. 2, 2004
(86) PCT No.: PCT/GB04/00865

§ 371 Date: May 8, 2006

(30) Foreign Application Priority Data
Mar. 4, 2003 (GB) 0304832.9

(51) Int. Cl. C12Q 1/70; C12Q 1/68

(52) U.S. Cl. 435/5; 435/6

(57) ABSTRACT

The use of a virus, which expresses and displays a binding moiety, as a means of detecting the presence or measuring the concentration of an analyte in a sample. The virus, which is typically a bacteriophage expressing a binding moiety, is used as a binding reagent in an immunoassay, and may be readily and accurately detected by detecting nucleic acid sequences of the virus.

The present invention provides a novel assay method as well as kits and reagents useful in said assay.

Immuno-PCR, which dates from 1992, uses nucleic acid tagged antibodies to provide a very sensitive assay endpoint. Generally the antibody is labelled with streptavidin and the nucleic acid through the streptavidin to the antibody. The biotinylated DNA is usually added at the end of the immunoassay.

Viruses may comprise essentially DNA or RNA, and attack host cells, integrating their nucleic acids into the host system. In structural terms, viruses generally express proteins which form a “coat” around the nucleic acids.

Phage display is a technique that was developed to allow proteins such as antibodies to be selected and produced in vitro. Phage libraries are made that contain a very high number of different proteins, such as scFv's (single chain variable fragments from antibodies). The phage has the DNA for the protein scFv's inserted into its genome and it expresses it as a fusion protein to the coat proteins, generally attached at its head.

The best protein such as scFv for a particular purpose is selected from the library mixture by “panning” for the phage that binds to the analyte of interest under strict selection conditions. The phage can then be multiplied by growth in its host bacterium and the DNA can be cut out and inserted into an expression vector. Binding protein can then be produced either as scFv or incorporated back into an antibody framework to make, for example, humanised antibodies for therapeutic use.

The phage display technique is thus used as an intermediate technique for in vitro antibody production. However, the phages themselves have never been proposed for use as assay reagents previously.

Thus according to the present invention there is provided the use of a virus, which expresses and displays a binding moiety, as a detectable moiety in an assay for detecting the presence or measuring the concentration of an analyte in a sample.

As used herein, the expression “detectable moiety” means that the virus itself is detected to provide a signal indicative of the presence or absence of an analyte.

In addition, the expression “binding moiety” relates to any moiety which will bind to a target, especially a polypeptide or protein, which may be for example an analyte polypeptide or protein, but may also be another polypeptide or protein, which is utilised in an immunoassay as part of the detection system.

The nucleic acid of the virus acts as a label, which may be detected using any of the known nucleic acid detection methods, in particular by using amplification reactions such as the polymerase chain reaction. However, the advantage of using a virus as compared to any other labelling technique is that a wide variety of binding moieties can be included relatively simply using techniques known for example for the production of recombinant viruses, such as phage display libraries, and without the need for additional binding steps.

Any type of virus may be used in the context of the invention, so that it may be a DNA virus, and in particular a bacteriophage (phage), which is a virus which attacks bacterial cells, but other DNA viruses or RNA viruses may also be employed. Thus, generally the virus will be a phage, and in particular a recombinant phage.

In particular, the virus will comprise a recombinant phage, which has been transformed so that expresses and displays a specific binding moiety such as an immunoglobulin for instance, an antibody, or a binding fragment thereof. However, the virus may be transformed to express any protein which may be of use in an immunoassay, including target analytes or analogues of these, even where these are not of the immunoglobulin superfamily.

Assay formats, which may use these reagents, may be any of the conventional assay forms known in immunology. For example, they may be used in both sandwich and competitive type assays.

Thus, the invention provides a method of detecting an analyte in a sample, said method comprising contacting said sample with a virus, which expresses and displays a binding moiety, such that the virus forms a complex with the analyte or an analogue thereof, or a binding partner for the analyte, and that the complex binds or does not bind a surface, depending upon the presence or absence of analyte in the sample, detecting the presence or absence of a nucleic acid on the surface, and relating that to the presence or absence of analyte in the sample.

The nucleic acid detected is suitably a nucleic acid which is characteristic of the virus, but there may be some assays formats where the presence of any nucleic acid will indicate that virus has been retained on the surface. In such cases, the nucleic acid may be detected for example using a dye, such as ethidium bromide which binds to DNA.

Suitably, the virus is incubated in the presence of the surface for a sufficient period of time to ensure that it binds to available binding sites on the surface, for example to any analyte present on the surface to form a bound analyte/virus complex, or to any binding reagent which is not occupied by analyte from the sample. For example, the incubation may take place for a period of from 5 to 60 minutes, at appropriate temperatures, such as from 25-40° C., for instance at about 37° C.

After this incubation, the surface including any immobilised complex is separated from the virus suspension, for example by removing the virus suspension and washing the surface.

Thereafter, a nucleic acid sequence, and in particular a nucleic acid, which may be a DNA or RNA, which is characteristic of the virus is detected on the surface.

In a sandwich type assay, the virus such as the phage is selected so that it will bind to an analyte within a sample to form a complex. A further binding reagent for the analyte is immobilised on a surface. When the sample is contacted with the surface in the presence of the virus, the complex of analyte and virus becomes bound to the surface. This may then be separated from the residual sample. Detection of viral nucleic acid retained on the surface is indicative of the presence of analyte within the sample.

In a typical competitive type assay, a binding reagent for an analyte, or the analyte or an analogue thereof, is immobilised on a surface.

As used herein, the expression “analogue” refers to a moiety that will bind to a binding partner which binds the analyte, even though it may not be of precisely the same sequence or structure as the analyte. It may, for instance, comprise a particular epitopic region of an analyte, which is bound by a specific monoclonal antibody, which therefore acts as the binding partner. Thus an analogue will “mimic” an analyte in the context of an immunoassay using a common binding partner.

A sample, to which a virus that expresses and displays a binding moiety for the analyte or analogue is added, is contacted with the surface. When analyte is present in the sample, it will compete with the immobilised analyte or analogue for binding to the virus. Thus, less virus will be retained upon the surface, than would be the case if no analyte was present in the sample. This reduction in the amount of retained virus can be detected in accordance with the invention, by analysing the surface for the presence of a nucleic acid present in the virus.

In an alternative competitive type assay, a binding reagent for the analyte is immobilised on the surface. In this case, the binding moiety displayed on the virus is a specific binding partner which is selected so that it competes with analyte for binding to the immobilised binding reagent. The less virus DNA detected on the surface after separation from the sample, the more analyte is present. Particular examples of analytes in this case are immunoglobulins such as antibodies, which may be useful in diagnosis of disease.

In all cases however, nucleic acid of the virus acts as detectable and specific “label” for the binding moiety, and may be detected using for example an amplification reaction such as a polymerase chain reaction or PCR reaction, which may be specific for the particular virus nucleic acid. Quantification of the analyte in the sample is possible using for example, quantitative PCR methods, as are well known in the art.

In a particular embodiment, the invention provides a method for detecting an analyte in a sample, said method comprising contacting a sample suspected of containing said analyte with a surface having immobilised thereon a binding reagent which either (a) binds said analyte, or (b) comprises said analyte or an analogue thereof, and a virus which expresses and displays a binding moiety that binds either said analyte or said binding reagent in competition to said analyte, separating said surface from the sample, and detecting the presence of a nucleic acid sequence present within said virus on said surface, wherein at least one of the binding reagent or the binding moiety is specific for the analyte.

In particular, as discussed above, both the immobilised binding reagent and the binding moiety binds the analyte. Where analyte is present in the sample, it will form a complex in the sample. The analyte also becomes bound to the binding reagent on the surface. When the surface is removed from the sample, bound analyte/virus complex will remain, and give a positive result when viral nucleic acid is assayed for. Conversely, where the sample does not contain target analyte, virus/binding moiety, which binds to that target analyte will not become associated with the surface, and so will not be detectable.

Alternatively, the immobilised binding reagent is an analogue of the analyte, which mimics the analyte in the sense that it will bind to the binding moiety of the virus in competition with the analyte. Thus the binding moiety will bind either the analyte or the binding reagent but not both. In this case, the sample is preferably incubated with the virus prior to contact with the surface. During this step, any analyte present will form a complex with the binding moiety on the virus, blocking the binding of the virus to the immobilised binding reagent on the surface. As a result, the complex will not be retained on the surface after washing and so the amount of detectable virus nucleic acid is reduced. In the absence of analyte, the binding moiety of the virus will be free to bind the binding reagent, resulting in a large viral nucleic acid “signal” being retained on the surface.

In some cases, the immobilised binding reagent binds the analyte, and also the binding moiety on the virus. In such cases, where the analyte is present in the sample, both the analyte and the binding moiety will compete for available sites on the surface. As a result, the amount of virus/binding moiety that is immobilised on the surface is reduced by an amount which relates to the concentration of analyte in the sample. Again, in this case, the presence of only a lower than expected “signal” from the viral nucleic acid is indicative of the presence of analyte in the sample.

This assay can be extremely sensitive, and background signals, which are associated with conventional immunoassay methods, can be reduced. The analysis itself is more readily controlled, as the virus can be engineered to comprise whatever sequence is convenient. The detection is entirely independent upon the nature of the analyte.

The binding reagent may be any reagent that will bind to analyte.

Analytes are generally proteins or polypeptides. Typical examples will be polypeptides or proteins that are associated with or part of a pathogenic organism such as a virus, bacteria or bacterial spore such as anthrax or anthrax spores, or a protein which is indicative of a particular disease state or of exposure to a particular disease, such as an immunoglobulin for instance an antibody.

Preferably, where the binding reagent binds the analyte, it is specific for the target analyte. However, it may be relatively non-specific, for example Protein A, where the target analyte is say an immunoglobulin such as IgG, provided that a binding moiety fused to the virus is specific for the target analyte.

Suitable specific binding reagents include antibodies or binding fragments thereof, as well as lectins. Antibodies may be monoclonal or polyclonal, but are preferably monoclonal.

The binding reagent is immobilised on the surface using conventional methods. For example Protein A may be used to bind antibodies or binding fragments which include the Fc region thereof.

The surface may be any convenient surface, such as the surface of a reaction plate or well, for instance an ELISA plate or well, in addition to beads such as magnetic beads, or membranes such as cellulose membranes which are used for example in dipstick assay tests, as are conventional in the art. Where appropriate, sites which are not occupied with binding reagent may be “blocked”, for example with protein such as bovine serum albumin or milk protein, or with polyvinylalcohol or ethanolamine, or mixtures of these, as is conventional in the art.

In a sandwich type assay, the sample is first incubated with the surface for a period sufficient to ensure that any analyte present becomes bound to the immobilised binding reagent. For example, the sample may be incubated with a blocked antibody-coated ELISA plate for a period of from 5 to 60 minutes, at appropriate temperatures, such as from 25-40° C., for instance at about 37° C.

The virus may be added prior to, during or after the incubation period. Preferably however, after the incubation, residual sample is removed from the surface, which is then washed to remove any unbound analyte, before the surface is then contacted with the virus.

The virus is suitably added in the form of a suspension. The surface is incubated with the virus suspension for a period of time sufficient to allow the virus to bind to analyte on the surface. After that, excess virus suspension is removed and the surface is washed before viral nucleic acid on the surface is detected. If necessary, the virus can be released from the surface, for example by boiling, before the detection reaction takes place.

In particular, the virus used in the method is a recombinant phage which expresses a binding moiety for the analyte or the binding reagent that is suitably a specific binding partner. In particular, the specific binding partner will comprise a single chain variable fragment of an antibody (scFV).

Recombinant phage expressing binding moieties may be produced using conventional methods, as is well known in the production of phage libraries for phage displays as discussed above (see for example Antibody Engineering, R. Konterman & S. Dubel (eds) Springer Lab Manuals, Springer-Verlag, Berlin Heidelberg, 2001). However similar techniques may be used to produce other types of recombinant viruses.

Viruses such as phages may be added singly or as multi-specificity mixtures, where more than one analyte is being looked for. In the latter case, detection of multiple nucleic acid sequences, each characteristic of individual viruses is carried out subsequently.

In one embodiment, the nucleic acid sequence of the virus is detected using an amplification reaction, for example a polymerase chain reaction (PCR). In this case, reagents, including primers, polymerases, nucleotides, and buffers as are well known, are added to the surface, and then subjected to thermal cycling as is conventional, in order to amplify any target nucleic acid sequence present.

The amplification product may then be detected using conventional methods such as gel electrophoresis, followed by visualisation using dyes.

Preferably the amplification reaction is carried out in such a way that the amplification product generates a detectable signal, and in particular a visible signal, for example a fluorescent signal, as it progresses. Many assay formats that produce such signals are known in the art. They may utilise reagents such as DNA binding agents such as intercalating dyes which emit radiation and particularly fluorescent radiation at greater intensity when they are intercalated into double stranded DNA, as well as probes and primers which include fluorescent labels, arranged to undergo fluorescent energy transfer (FET) and particularly fluorescent resonant energy transfer (FRET).

There are two commonly used types of FET or FRET probes, those using hydrolysis of nucleic acid probes to separate donor from acceptor, and those using hybridisation to alter the spatial relationship of donor and acceptor molecules.

Hydrolysis probes are commercially available as TaqMan™ probes. These consist of DNA oligonucleotides that are labelled with donor and acceptor molecules. The probes are designed to bind to a specific region on one strand of a PCR product. Following annealing of the PCR primer to this strand, Taq enzyme extends the DNA with 5′ to 3′ polymerase activity. Taq enzyme also exhibits 5′ to 3′ exonuclease activity. TaqMan™ probes are protected at the 3′ end by phosphorylation to prevent them from priming Taq extension. If the TaqMan™ probe is hybridised to the product strand, an extending Taq molecule may also hydrolyse the probe, liberating the donor from acceptor as the basis of detection. The signal in this instance is cumulative, the concentration of free donor and acceptor molecules increasing with each cycle of the amplification reaction.

Hybridisation probes are available in a number of forms. Molecular beacons are oligonucleotides that have complementary 5′ and 3′ sequences such that they form hairpin loops. Terminal fluorescent labels are in close proximity for FRET to occur when the hairpin structure is formed. Following hybridisation of molecular beacons to a complementary sequence the fluorescent labels are separated, so FRET does not occur, and this forms the basis of detection.

Pairs of labelled oligonucleotides may also be used. These hybridise in close proximity on a PCR product strand-bringing donor and acceptor molecules together so that FRET can occur. Enhanced FRET is the basis of detection. Variants of this type include using a labelled amplification primer with a single adjacent probe.

Other methods for detecting amplification reactions as they occur are known however, and any of these may be used. Particular examples of such methods are described for example in WO 99/28500, British Patent No. 2,338,301, WO 99/28501 and WO 99/42611.

WO 99/28500 describes a very successful assay for detecting the presence of a target nucleic acid sequence in a sample. In this method, a DNA duplex binding agent and a probe specific for said target sequence, is added to the sample. The probe comprises a reactive molecule able to absorb fluorescence from or donate fluorescent energy to said DNA duplex binding agent. This mixture is then subjected to an amplification reaction in which target nucleic acid is amplified, and conditions are induced either during or after the amplification process in which the probe hybridises to the target sequence. Fluorescence from said sample is monitored.

An alternative form of this assay, which utilises a DNA duplex binding agent which can absorb fluorescent energy from the fluorescent label on the probe but which does not emit visible light, is described in co-pending British Patent Application No. 223563.8. Any of these assays may be used in the context of the assay method of the invention in order to detect the target nucleic acid sequence.

Many of these assays can be carried out in a quantitative manner as is well known in the art, for example by monitoring the signal from the amplification mixture at least once during each cycle of the amplification reaction. By carrying out the reaction in this way, the amount of virus present on the surface may be determined, and this may be related to the amount of analyte present in the original sample.

The particular sequence of the virus detected may be any characteristic sequence found therein. Where single specificity viruses are used in the assay, this may be any sequence found within the phage itself, as well as the sequence encoding the complementarity determining region (CDR) of the scFv, the “scaffolding” for the CDR of any recombinant virus, or other sequences introduced into the recombinant virus during its preparation such as antibiotic resistance genes.

If desired, specific marker sequences may be included in the virus in addition to those coding for the binding moiety. They may be introduced into the virus at the same time as the binding moiety, for example fused to the sequence encoding the binding moiety, or may be added in a separate transformation operation.

Where multi-specificity mixtures of viruses are used in the assay, then it is necessary to detect sequences which are characteristic of each particular virus, in order to determine whether specific analytes are present in the sample. In this case therefore, it is necessary to detect sequences such as the sequence encoding the scFv itself, or a specifically introduced marker sequence, as discussed above.

In this case, sequences common to viruses or recombinant viruses, such as phage DNA or RNA, or antibiotic resistance genes, may also be detected. Generally, there will always be some carry-over of viral nucleic acid, which can be used as internal reference sequences, ensuring that the PCR reaction has proceeded appropriately.

In this case multiplex PCR reactions using different signalling reagents or systems may be employed in order to detect the various sequences which are produced. This may be achieved, for example by labelling probes or primers used in the amplification reaction using different labels, for example, labels which fluoresce at different wavelengths. Examination of the signal from each label, for example at each of the different wavelength, is then carried out, if necessary with appropriate signal resolution where the wavelengths overlap.

Alternatively the assay is designed such that the amplicons produced by different PCR reactions hybridise to form duplexes or destabilise at different temperatures. Melting point analysis, for example using intercalating dyes that exhibit increased fluorescence when bound to double stranded DNA species, is a well-known technique. By adding such as dye to the reaction system, either during or after the assay process, and by monitoring fluorescence with a controlled change of temperature, the temperature at which the duplex structure of the amplicon breaks down or reforms can be determined, and this can be related to the presence of the particular amplicon and hence the particular virus which has bound.

The assay system of the invention thus provides a useful and reliable assay method.

Kits for use in the assay method described above form a further aspect of the invention.

In particular, the invention provides kit for detecting the presence of an analyte in a sample, said kit comprising solid body having immobilised on a surface thereof a binding reagent which either (a) binds said analyte, or (b) comprises said analyte or an analogue thereof, and a virus, such as a recombinant phage, which expresses and displays a binding moiety either said analyte or said binding reagent in competition to said analyte.

For instance, where the surface has immobilised thereon a binding reagent which binds said analyte, the virus suitably expresses and displays a binding partner for the analyte, or a binding partner which binds said binding reagent in competition to said analyte.

Alternatively, where the surface has immobilised thereon a binding reagent which comprises either the analyte or an analogue thereof, the virus is suitably one which expresses and displays a binding partner for said analyte.

Suitably, the solid body is a well in a plate, for instance a multi-well plate, but it may also be beads such as magnetic beads, or membranes, for example cellulose membranes as found in conventional dipstick type assays.

The kit may include more than one type of virus, in particular recombinant phages, for use in multi-specificity assays as discussed above.

Possible additional elements of the kit comprise reagents suitable for use in the detection of the nucleic acid sequences. In particular, the kit may comprise intercalating dyes, primers or probes for use the detection of the particular nucleic acid sequences as discussed above. For example, the kit may comprise primers which amplify sequences, which encode specific scFv sequences, or marker sequences which have been incorporated into the virus. In addition, or alternatively in the case of single specificity assays, the kits may include primers which are suitable for amplifying virus sequences, sequence which encode scaffolding of scFvs or antibiotic resistance genes which are present in recombinant virus.

The primers may suitably be labelled in such a way that the amplification product is directly detectable. For example, they may include fluorescent or other labels as described above.

Additionally or alternatively, the kit may include probes, which are specific for the amplification product and which are labelled to assist in detection of product. They may comprise single- or dual-labelled hydrolysis or hybridisation probes, also as discussed above. When appropriate they may include intercalating dyes or other DNA duplex binding agents, which form elements of the detection system.

Kits may also include intercalating dyes to assist with melting point analysis, where this is required in order to resolve multi-specificity assay results.

Recombinant viruses and in particular recombinant phages, which are transformed so that they express both a binding moiety and a marker sequence, are novel and as such form a further aspect of the invention.

The invention will now be particularly described by way of Example with reference to the accompanying drawings in which:

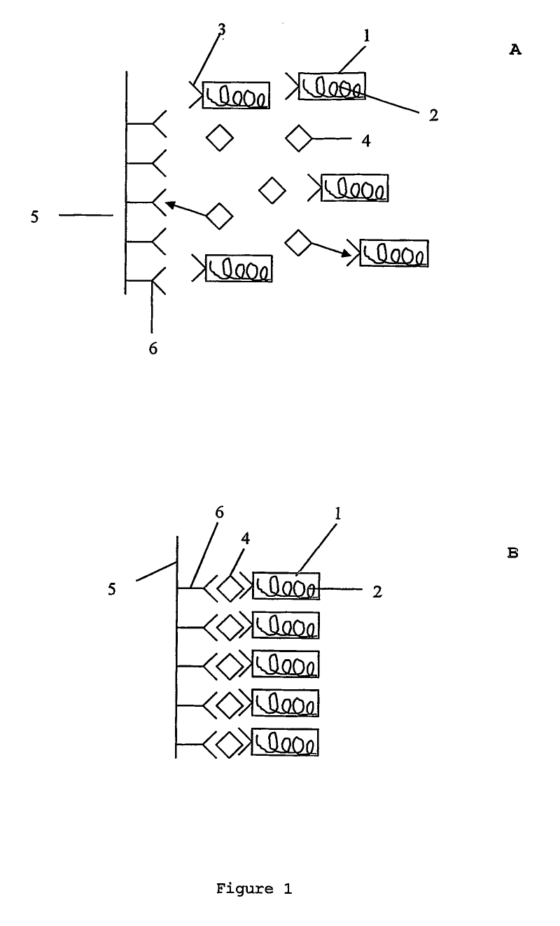

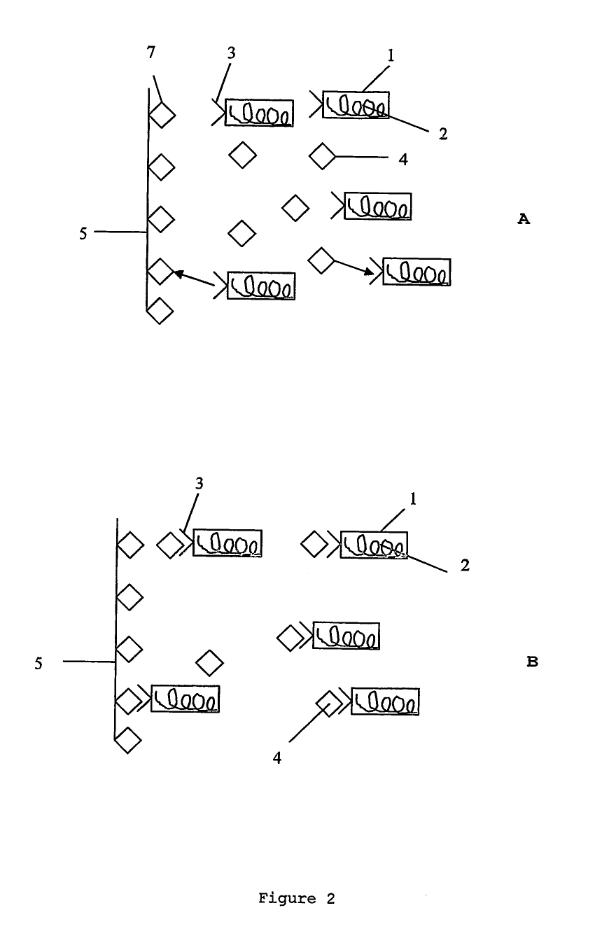

FIG. 1 illustrates diagrammatically, a sandwich assay including the invention;

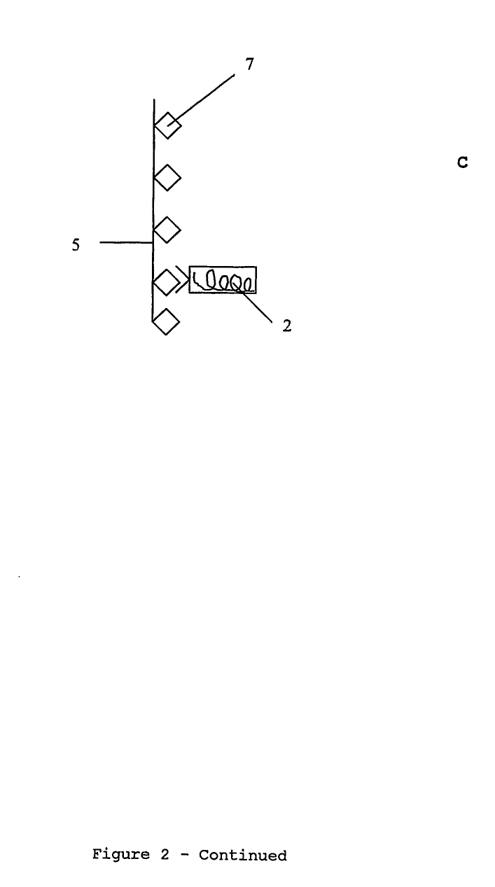

FIG. 2 illustrates diagrammatically a competitive assay including the invention;

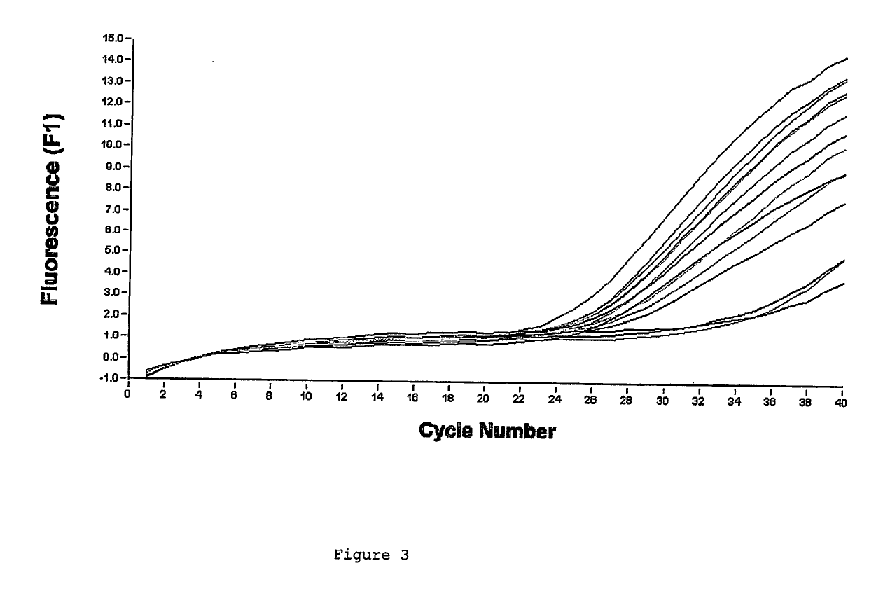

FIG. 3 is a graph of fluorescence −d(F1)/dt versus temperature when carrying out a PCR reaction of the TAQMAN® type;

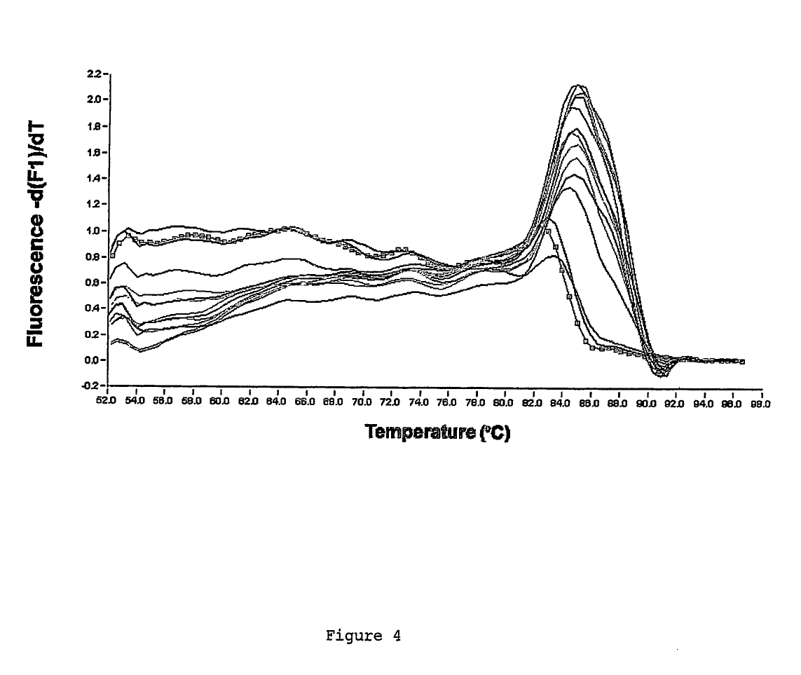

FIG. 4 is a graph of fluorescence (F1) against cycle number of series of samples at different dilutions using the assay of the invention; and

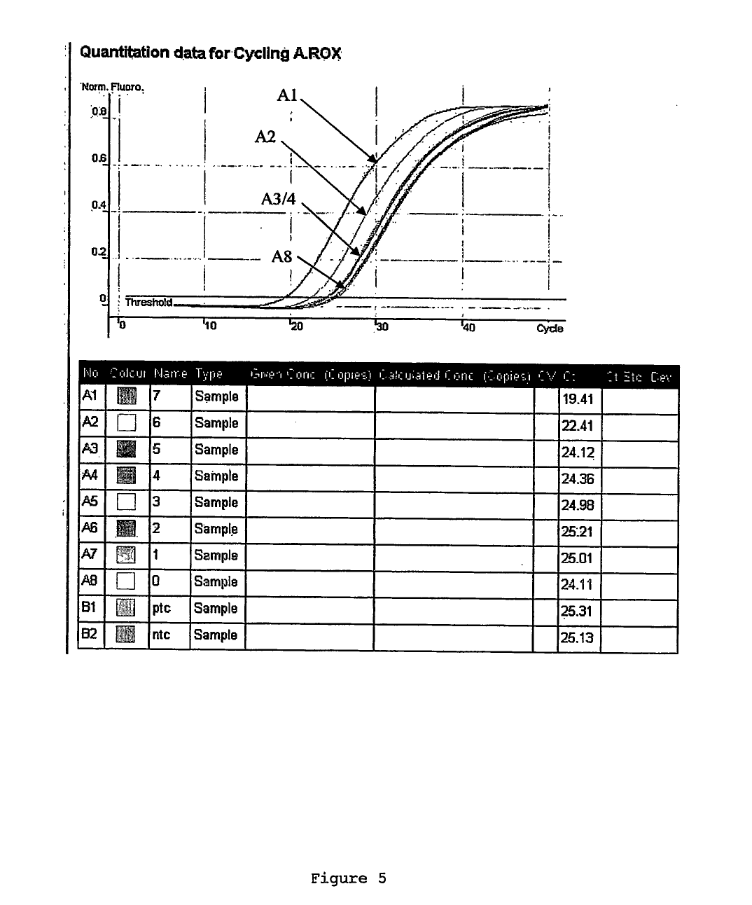

FIG. 5 shows the results of an assay described hereinafter for B. cereus spores including a PCR for detecting phage DNA.

In the sandwich assay illustrated in FIG. 1A, a phage (1) is used, which comprises an outer coat, enclosing phage DNA (2). A binding partner (3) such as an scFv is expressed by the phage and displayed on the surface at the head of the phage. It is added as a reagent to an assay reaction mixture which may contain analyte (4), and is in contact with a surface, such as a bead or well (5) on which an antibody (6) which is also specific for the analyte (4) is immobilised.

On incubation (B), the phage (1) and analyte (4) forms a complex which is retained on the surface (5), by the binding of the analyte to the antibody (6). Thereafter, the surface (5) is removed from the remainder of the sample and washed. However, some phage is retained on the surface, where it may be detected.

In the embodiment illustrated in FIG. 2A, an analyte or an analogue thereof (7) capable of binding to the binding partner (3) of the phage (1) is immobilised on the surface (5). A sample under test to which the phage (1) has been added is incubated in the presence of this surface. Analyte (4) in the sample will bind to the binding partner (3) of the phage (1). Any phage which has undergone such binding is unable to bind to the immobilised analyte analogue (7) (B), and therefore will be washed away with the sample during a subsequent separation step. Detection of phage DNA (2) on the surface (5) following such a washing step will reveal lower levels than would otherwise be expected if no analyte were present.

Other assay formats are possible as would be understood in the art.

### EXAMPLE 1

Demonstration of Assay Using Bacillus cereus Spores

1) Plate Format

A sample (50 μl) comprising a suspension of B. cereus spores (1×10⁸ per ml) in distilled sterile water was added to each well of a blocked ELISA plate (Immulon microtitre ELISA plate) which was then placed in an oven at 37° C. overnight to dry the spores onto the plates.

The plates were then washed three times with a wash solution comprising 0.05% v/v Tween 20 in phosphate buffered saline (PBS) or PBST.

A blocking buffer (200 ml) comprising 2% w/v dry milk powder and 0.05% v/v Tween 20 PBS was added to each well. The plate was then sealed and incubated at room temperature for a minimum of 1 hour. It was then washed again three times in PBST.

A solution of primary antibody expressing M13 phage (1×10⁹ transforming units per ml), wherein the primary antibody is a B. cereus specific single chain variable fragment (scFv), in PBST blocking buffer was prepared and at least 50 μl added per well.

PBST blocking buffer was added to one of the wells in place of the primary antibody expressing phage as a negative control.

The plate was incubated at 37° C. for 1 hour, then washed 5 times in PBST. After drying, 50 μl dH₂O was added to each well. The plate was then boiled for 30 seconds to free the phage for PCR. After allowing the plate to cool briefly, and contents of the wells were transferred to a PCR tube, together with a conventional PCR mix (18 μl) including M13 phage specific primers and SybrGreen, used in accordance with the manufacturer's instructions.

The sample subjected to a series of thermal cycling steps on the Roche LightCycler as follows:

94° C. for 0 seconds (melt);

62° C. for 30 seconds (annealing phase);

72° C. for 30 seconds (extension phase).

40 cycles were carried out. The fluorescent signal from the samples was monitored once per cycle at the end of the extension phase. The process was repeated with an increasingly dilute sample and the results are shown in FIG. 3.

Negative control samples showed only a small increase in signal at the end of the cycling process. It was confirmed by a final melt curve analysis (FIG. 4) that the signal from the negative control was due to non-specific products such as primer-dimers.

The results show however that the presence of bacterial spores in the samples was detectable using this method.

### EXAMPLE 2

Detection Assay

For use as a detection assay, a sample is added to a blocked-antibody coated ELISA plate and incubated at 37° C. for 5 to 60 minutes.

Thereafter, the plate is washed with wash liquid from three to five times.

A suspension of filamentous phage expressing an scFv specific for the assay target is added to the plate, and incubated for 5-60 mins. After further washing (3-5 times), conventional PCR reagents are added, together with a suitable reporter system such as the SybrGreen dye mentioned above. However, other reporter mechanisms, for example using fluorescent reporter probes, such as TAQMAN® or other probes for in situ monitoring may be employed. The reaction mixture is thermally cycled to effect the amplification in the conventional way.

The PCR cycle number at which product appears (fluorescence threshold crossing point) is noted and correlated with the concentration of analyte in original sample.

It is possible to add more than one scFv with different specificities at the same time, to determine a range of targets. In such cases, melt profiles may be carried out to distinguish which one of the scFv is present and therefore has bound to the analyte. If desired, phage sequences or antibiotic resistance sequences found in the transformed phage may be used as an internal reference for the PCR.

### EXAMPLE 3

Alternative Filtration Assay Format

In this embodiment, a liquid sample is passed through a 0.2 or 0.45 micron filter, depending upon the nature of the assay target, and the filter is then washed. Target analyte, for example bacterial spores, are retained on the filter. Subsequently, a suspension of filamentous phage expressing an scFv specific for the assay target is also passed through the filter, which is again washed. Any phage which binds to the target on the filter can then be detected, by PCR as described above.

### EXAMPLE 4

Detection of Phage Displaying Single Chain Antibodies Directed Against B cereus in an Immunoassay Format.

50 μl of 107 B. cereus spores/ml were diluted 10 fold in dH₂O down an Immulon 2 ELISA plate spores and dried onto the plate at 37° C. overnight. This immobilised the spores on the plate so that they mirrored the situation in which an analyte was binding an immobilised antibody, as might occur in an assay for the spores.

The plate was then washed in dH₂O, three times. Each well was then blocked by the addition of 150 μl of 1% blotto/phosphate buffered saline (PBS) and the plate was then incubated at 37° C. for 1 hour. The plate was washed in PBS-Tween, three times. 50 μl of phage suspension in 1% blotto/PBS was added to each well and then the plate was incubated at 37° C. for one hour, so that the phage bound to the spores on the plate. The plate was then washed in PBS-Tween 3 times, followed by three washes in dH₂O to remove unbound phage.

50 μl of dH₂O was then added to each well and the plate was heated in boiling water for 30 seconds so as to elute the phage.

2 μl from each well were then assayed by PCR using phage directed primers, amplify the lacI gene found within the M13 derived phage. Real-time PCR was performed using a Corbett RotorGene. Each tris-buffered reaction contained 0.5 μM each of forward and reverse LacI primers, 0.3 μM of lacI specific TaqMan probe, 4 mM MgCl₂. Cycling parameters were 95° for 5 seconds and 60° C. for 1 minute for 50 cycles. The primers, probe and target were as follows:

| | |
|---|---|
| FORWARD PRIMER | 5′-CGTGGTGGTGTCGATGGTAG |
| REVERSE PRIMER | 5′-TGTGCACCGCTTT |
| PROBE SEQUENCE | 5′-ACGAAG CGGCGTCGAA GCCTG |
| AMPLICON | 5′-CGTGGTGGTGTCGATGGTAGAACGA AGCGGCG |
| | TCGAAGCCTGTAAGCGGCGGTGCACA |

The results are shown in FIG. 5. The results show that 10⁶ to 10³ spores per well were detectable above background level, even though the background level in this case was quite high. This was probably as a result of cross-contamination. There are shared genes between M13 derived phages and M13 derived cloning vectors used routinely in the lab. The sensitivity of the assay could be readily improved by setting up the phage PCR reaction in a lab that is free of M13 contamination or by choosing primers specific to phages displaying B. cereus antibodies.

1-6. (canceled)

7. A method of detecting an analyte in a sample, said method comprising contacting said sample with a virus, which expresses and displays a specific binding moiety, such that the virus forms a complex with the analyte or an analogue thereof, or a binding moiety for the analyte, and that the complex binds or does not bind a surface, depending upon the presence or absence of analyte in the sample, detecting the presence or absence of a nucleic acid on the surface using a nucleic acid amplification reaction, and relating that to the presence or absence of analyte in the sample.

8. The method of claim 7 which comprises contacting the sample suspected of containing an analyte with a surface having immobilised thereon a binding reagent which either (a) binds said analyte, or (b) comprises said analyte or an analogue thereof, and a virus which expresses and displays a binding moiety that binds either said analyte or said binding reagent in competition to said analyte, separating said surface from the sample, and detecting the presence of a nucleic acid sequence present within said virus on said surface, wherein at least one of the binding reagent or the binding moiety is specific for the analyte.

9. The method of claim 8 where both the immobilised binding reagent and the binding moiety binds the analyte, so that a complex comprising the binding reagent, the analyte and the virus is retained on the surface after separation of the sample therefrom, and the presence of viral nucleic acid on the surface is indicative of the presence of analyte in the sample.

10. The method of claim 8 where the immobilised binding reagent comprises the analyte or an analogue thereof, so that the binding moiety will bind either the analyte or the binding reagent, and the presence of analyte in the sample blocks the binding of the binding moiety to the binding reagent, so that a reduction in the amount of virus able to bind to the binding reagent is indicative of the presence of analyte in the sample.

11. The method of claim 8 wherein the binding reagent binds the analyte, and also the binding moiety on the virus, so that the analyte and the binding moiety will compete for available sites on the surface, so that a reduction in the amount of virus able to bind to the binding reagent is indicative of the presence of analyte in the sample.

12. The method of claim 8 wherein the binding reagent is a specific binding reagent.

13. The method of claim 7 wherein the surface is the surface of an ELISA plate or well, a magnetic bead or a membrane.

14. The method of claim 13 wherein sites on the surface which are not occupied with binding reagent are blocked.

15. The method of claim 9 in which the following steps are carried out sequentially:
i) sample is incubated with the surface for a period sufficient to ensure that any analyte present becomes bound to the immobilised binding reagent,
ii) residual sample is removed from the surface, which is then washed to remove any unbound analyte,
iii) the surface is contacted with a suspension of the virus, and incubated for a period of time sufficient to allow the virus to bind to analyte on the surface;
iv) virus suspension is removed and the surface is washed; and nucleic acid of the virus on the surface is detected.

16. The method of claim 15 wherein the virus nucleic acid is released from the surface before step (v).

17. The method of claim 7 wherein the virus is a recombinant phage which has been transformed so that it expresses a specific binding moiety which specifically binds either the analyte or a specific binding partner for the analyte.

18. The method of claim 17 wherein the specific binding partner is a single chain variable fragment of an antibody (scFV).

19. The method of claim 7 wherein the virus comprises a multi-specificity mixture.

20. The method of claim 19 wherein detection of multiple nucleic acid sequences, each characteristic of individual viruses, is carried out.

21. The method of claim 7 wherein the nucleic acid amplification reaction is a polymerase chain reaction (PCR).

22. The method of claim 21 wherein the amplification reaction is carried out in such a way that the amplification product generates a detectable signal.

23. The method of claim 22 wherein the signal is a visible signal.

24. The method of claim 22 or claim 23 wherein the amplification reaction is carried out in the presence of a DNA binding reagent, or a primer or probe which is labelled with a fluorescent label.

25. The method of claim 24 wherein the DNA binding agent is an intercalating dye.

26. The method of claim 7 wherein the amount of virus detected is quantified, and this is related to the amount of analyte in the sample.

27. The method of claim 7 wherein virus nucleic acid is detected by amplifying a nucleic acid sequence which is characteristic of a particular virus used in the method.

28. The method of claim 27 wherein the virus is a recombinant virus which has been transformed with a marker sequence, and this sequence is the sequence which is amplified.

29. The method of claim 27 wherein sequences characteristic of more than virus are detected.

30. The method of claim 7 where more than one virus is used in the process, and a subsequent melting point analysis is conducted to determine which virus has bound during the assay.

31. A kit for detecting the presence of an analyte in a sample, said kit comprising solid body having immobilised on a surface thereof a binding reagent which either (a) binds said analyte, or (b) comprises said analyte or an analogue thereof, and a virus which expresses a specific binding moiety for either said analyte or said binding reagent in competition to said analyte.

32. The kit of claim 31 wherein the virus is a recombinant phage which expresses a specific binding moiety for either said analyte or said binding reagent in competition to said analyte.

33. The kit of claim 31, which comprises more than one type of recombinant virus.

34. The kit of claim 31, which further comprises reagents suitable for use in the amplification of nucleic acid sequences of the said virus.

35. The kit of claim 31, which further comprises an intercalating dye.


SEQUENCE LISTING (4 sequences)

SEQ ID NO: 1 - DNA, 20 bp, Artificial Sequence
  Forward Primer
cgtggtggtgtcgatggtag

SEQ ID NO: 2 - DNA, 13 bp, Artificial Sequence
  Reverse Primer
tgtgcaccgcttt

SEQ ID NO: 3 - DNA, 21 bp, Artificial Sequence
  Probe
acgaagcggcgtcgaagcctg

SEQ ID NO: 4 - DNA, 58 bp, Artificial Sequence
  M13 Derived Phage
cgtggtggtgtcgatggtagaacgaagcggcgtcgaagcctgtaagcggcggtgcaca